(19) United States
(12) Patent Application Publication
(10) Pub. No.: US 2020/0283714 A1
(43) Pub. Date: Sep. 10, 2020

(54) AEROBIC ORGANIC SOLID MATERIAL PROCESSOR

(71) Applicant: James BOMFORD, Cowichan Bay (CA)

(72) Inventor: James BOMFORD, Cowichan Bay (CA)

(21) Appl. No.: 16/799,919

(22) Filed: Feb. 25, 2020

(30) Foreign Application Priority Data
Mar. 4, 2019 (CA) 3035573

(51) Int. Cl. C12M 1/06; C12M 1/00

(52) U.S. Cl. CPC C12M 27/06; C12M 29/00; C12M 23/34

(57) ABSTRACT

An aerobic organic material processor includes a tank having an outer wall, a top, a bottom, a first end, a second end, and a horizontal axis. The tank is divided into compartments distributed along the horizontal axis of the tank between the first end of the tank and the second end of the tank. Each compartment is connected to other compartments by a fluid passageway toward the bottom of the tank. An agitator is provided for agitating the organic material within the tank. Fluid ports are positioned toward the top of the tank in communication with each of the compartments. Applying either a suction force or pressure to at least one of the fluid ports causes fluids to migrate from compartment to compartment via the fluid passageway, thereby aerating organic material within the tank.

### FIELD

The present application relates to an organic material processor that uses oxygen to compost organic material aerobically.

### BACKGROUND

Organic waste processing units that aerobically convert waste to compost are known. A problem common to all aerobic organic waste processors, as opposed to anaerobic organic waste processors, is the circulation of oxygen. Fluids, including oxygen, tend to “short circuit”, through the organic material following a path of least resistance. U.S. Pat. No. 4,042,219 (Terry) discloses one way of introducing oxygen into an aerobic organic waste processor.

### SUMMARY

There is provided an aerobic organic material processor which includes a tank having an outer wall, a top, a bottom, a first end, a second end, and a horizontal axis. The tank is divided into compartments distributed along the horizontal axis of the tank between the first end of the tank and the second end of the tank. Each compartment is connected to other compartments by a fluid passageway toward the bottom of the tank. An organic material input is positioned toward the first end of the tank and an organic material output is positioned toward the second end of the tank. An agitator is provided for agitating the organic material within the tank. Fluid ports are positioned toward the top of the tank in communication with each of the compartments. The tank can be operated in either a suction mode or a pressurized mode as will hereafter be further described.

### BRIEF DESCRIPTION OF THE DRAWINGS

These and other features will become more apparent from the following description in which reference is made to the appended drawings, the drawings are for the purpose of illustration only and are not intended to be in any way limiting, wherein:

FIG. 1 is a first side elevation view, in section, of an aerobic organic material processor with arrows showing a path of air through the processor.

FIG. 2 is a second side elevation view, in section, of the aerobic organic material processor illustrated in FIG. 1, with arrows showing a path of organic material through the processor.

FIG. 3 is an end elevation view in section of the aerobic organic material processor illustrated in FIG. 1.

### DETAILED DESCRIPTION

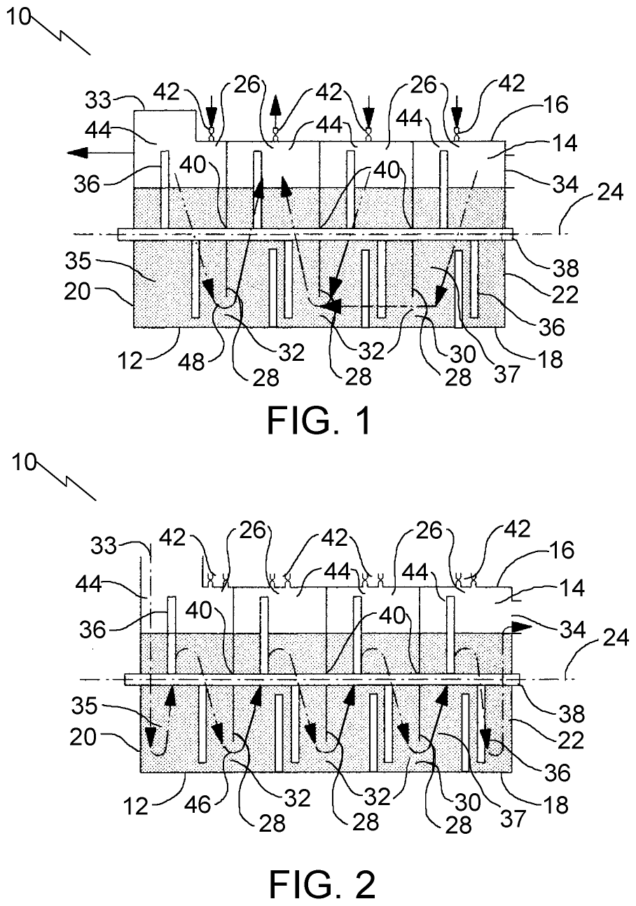

An aerobic organic material processor, generally identified by reference numeral 10, will now be described with reference to FIG. 1 through 3.

Structure and Relationship of Parts:

Referring to FIG. 1, aerobic organic material processor 10 includes a tank 12 having an outer wall 14, a top 16, a bottom 18, a first end 20, a second end 22, and a horizontal axis 24. Tank is preferably a static and corrosion resistant, insulated, sealed, horizontally oriented, cylindrical vessel. Tank 12 is divided into individual, sealed compartments 26 by baffles 28 sealably connected to outer wall 14, although other means of creating sealed compartments 26 will be recognized by those skilled in the art. Compartments 26, preferably 3 or more, are distributed along horizontal axis 24 of tank 12 between first end 20 and second end 22. Referring to FIG. 1 through 3, each compartment 26 is fluidly connected to other compartments 26 by a fluid passageway 30 toward bottom 18 of tank 12. Passageway 30 is formed from openings 32 in baffles 28 toward bottom 18 of tank 12. Referring to FIG. 1, an organic material input 33 is positioned toward first end 20 of tank 12, and an organic material output 34 is positioned toward second end 22 of tank 12. Organic material 35 enters tank 12 through input 33, and is removed, after processing, from output 34. The processing elements will be discussed below.

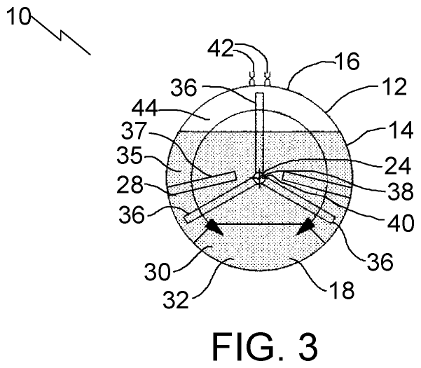

Referring to FIG. 3, agitators are provided in the form of arms 36 that depend from a rotatable shaft 38. Arms 36 are positioned in each compartment 26 for agitating the organic material within tank 12. Arms act to macerate, mix, aerate and move organic material 35. Referring to FIG. 1, Shaft 38 extends substantially parallel to axis 24 of tank 12 through baffles 28. Each baffle 28 has a sealed connection 40 for receiving shaft 38. Sealed connection 40, in addition to the sealed connection of baffles 28 to outer wall 14, prevents the circulating gas from “short circuiting” fluid passageway 30, while still permitting rotation of shaft 38. The movement of shaft 38 is controlled by a motor (not shown) which can rotate shaft 38 in a variety of ways. Rotation may be continuous, intermittent, in one direction only, or it may change directions, to ensure that organic material 35 is well mixed. Organic material may be made up of biodegradable organic material and other amendments such as carbonaceous solids, such as wood chips, straw, peanut shells, etc, and other materials. The preferred state of the carbonaceous amendments is in a dried, pellet form.

While rotating arms 36 function adequately as agitators in most situations, it was discovered that under certain conditions organic material 35 turned with rotating arms 36. When this occurred, the mixing was less than desired. To address this problem, fixed arms 37 were secured to outer wall 14 of tank 12 and interposed between rotating arms 36. Fixed arms 37 resist rotation and disrupt organic material 35, thereby improving the mixing. Referring to FIG. 3, the positioning of fixed arms 37 spaced from the bottom of tank 12 should be noted. If fixed arms 37 were centered on the bottom of tank 12, they likely would interfere with material flow. Referring to FIG. 1 and FIG. 2, fixed arms 37 are preferably centered in compartments 26 between baffles 28.

Fluid ports 42 toward top 16 of tank 12 are provided that are in communication with each compartment 26. A gas cavity 44 is maintained about each fluid port 42 within tank 12 to prevent organic material 35 from blocking fluid ports 42. Fluid ports 42 may be used to supply air or remove air. By conditioning the air that is supplied relative to the gas that is removed, the temperature, humidity, and aeration or oxygen content of organic material 35 can be controlled.

Operation:

The use of aerobic organic material processor 10, as described above with reference to FIG. 1 through 3, will now be discussed. Organic material 35 is added to tank 12 through input 33, and removed through output 34, each of which may be done continuously or intermittently. As tank fills with organic material 35, each compartment 26 will be filled, with a gas cavity 44 remaining toward top 16 of tank 12 in each compartment 26 that prevents material in tank 12 from blocking fluid ports 42. Organic material 35 is agitated by rotating shaft 38 and thus arms 36, to cause organic material 35 to be macerated, mixed, aerated, and moved through tank 12. As described above, the addition of fixed arms 37 is recommended as it improves mixing of organic material 35. The operation of shaft 38 may be timer and/or temperature controlled. Organic material 35 makes its way from input 33 toward output 34 by passing under baffles 28 through passageway 30. Referring to FIG. 2, arrows 46 show a simplified path that organic material 35 takes as it passes through tank 12. To aerate organic material 35, it is also necessary to draw air through tank 12, and thus through organic material 35 in tank 12. To do so, a suction force is applied to one or more of the fluid ports 42 such that gas is extracted from the corresponding compartment 26. This in turn causes air to be drawn through other fluid ports 42 into other compartments 26. In this context, “air” includes oxygen, outside air, water vapour, or any combination thereof, or other additives that it may be desired to inject into tank 12. The air migrates through fluid passageway 30, as the rest of baffle 28 is sealed to prevent air from “short circuiting” passageway 30. Referring to FIG. 1, arrows 48 show a possible path taken by the air in tank 12 as it passes through organic material 35. By conditioning air that enters ports 42 relative to gas being drawn from ports 42, it is possible to control the temperature, oxygen level and the moisture level in each compartment. The temperature, oxygen levels and moisture level are determined as is known in the art. In addition, applying a vacuum also has the effect of removing harmful gases to prevent any dangerous situations. Air is drawn into and out of different compartments 26 at different times based on the need to regulate temperature, oxygen and moisture of organic material 35. Alternatively, other fluids besides air or oxygen, such as water may be injected directly through fluid ports 42.

### Variation on the Mode of Use

Tank 12 can also be operated in a pressurized mode, instead of the suction mode described above. In the pressurized mode, air under pressure is injected through one of more of the fluid ports. This causes gas to be expelled from the other fluid ports. The suction mode is the preferred operating mode. The reason for this is that it provides better control over noxious fluids that would otherwise be exiting from multiple ports. There will, however, be situations in which it is preferable to inject fluids under pressure.

In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be one and only one of the elements.

It will be apparent to one skilled in the art that modifications may be made to the illustrated embodiment without departing from the spirit and scope defined in the Claims.

What is claimed is:

1. An aerobic organic material processor, comprising:
a tank having an outer wall, a top, a bottom, a first end, a second end, and a horizontal axis, the tank being divided into compartments distributed along the horizontal axis of the tank between the first end of the tank and the second end of the tank, each compartment being fluidly connected to other compartments by a fluid passageway toward the bottom of the tank;
an organic material input positioned toward the first end of the tank;
an organic material output positioned toward the second end of the tank;
an agitator for agitating the organic material within the tank; and
fluid ports toward the top of the tank in communication with each of the compartments.

2. The aerobic organic material processor of claim 1, wherein the compartments are formed from baffles sealably connected to the outer wall of the tank.

3. The aerobic organic material processor of claim 2, wherein the fluid passageway comprises an opening in the baffle toward the bottom of the tank.

4. The aerobic organic material processor of claim 1, wherein the agitator depends from a rotatable shaft extending substantially parallel to the axis of the tank

5. The aerobic organic material processor of claim 4, wherein the shaft is driven by a motor.

6. The aerobic organic material processor of claim 4, wherein the agitator comprises rotating arms depending from the rotatable shaft positioned in each compartment, and fixed arms affixed to the outer wall of the tank and interposed between the rotating arms.

7. The aerobic organic material processor of claim 4, wherein the compartments are formed from baffles sealably connected to the outer wall of the tank, each baffle having a sealed connection for receiving the shaft, the sealed connection permitting rotation of the shaft.

8. An aerobic organic material processor, comprising:
a tank having an outer wall, a top, a bottom, a first end, a second end, and a horizontal axis, the tank being divided into compartments by baffles sealably connected to the outer wall, distributed along the horizontal axis of the tank between the first end of the tank and the second end of the tank, each compartment being fluidly connected to the other compartments by a fluid passageway toward the bottom of the tank, the passageway comprising an opening in the baffle toward the bottom of the tank;
an organic material input positioned toward the first end of the tank;
an organic material output positioned toward the second end of the tank;
rotating arms depending from a rotatable shaft positioned in each compartment for agitating the organic material within the tank, and fixed arms affixed to the outer wall of the tank and interposed between the rotating arms, the shaft extending substantially parallel to the axis of the tank, the shaft being driven by a motor, each baffle having a sealed connection for receiving the shaft, the sealed connection permitting rotation of the shaft; and
fluid ports toward the top of the tank in communication with each of the compartments.

9. A method of introducing oxygen into an aerobic organic material processor, comprising the steps of:
providing an aerobic organic material processor, comprising:
a tank having an outer wall, a top, a bottom, a first end, a second end, and a horizontal axis, the tank being divided into compartments distributed along the horizontal axis of the tank between the first end of the tank and the second end of the tank, each compartment being fluidly connected to the other compartments by a fluid passageway toward the bottom of the tank;
an organic material input positioned toward the first end of the tank;
an organic material output positioned toward the second end of the tank;
an agitator for agitating the organic material within the tank; and
providing fluid ports toward the top of the tank in communication with each of the compartments;
applying a suction force to one of the fluid ports to extract gas from at least one of the compartments and continuing to apply suction to cause air to be drawn through other fluid ports into other compartments and migrate through the fluid passageway, thereby aerating organic material within the tank.

10. The method of claim 9, including a step of maintaining a gas cavity about each fluid port within the tank to prevent the organic material from blocking the fluid ports and to provide fluids access to the full surface area of the material contained within the compartments.

11. The method of claim 9, including a step of using the fluid ports to one of inject gases and water vapour into the tank or remove gases and water vapour from the tank.

12. The method claim 11, gases and water vapour being injected or removed from the tank to control at least one of the temperature, the oxygen level and the moisture level in one or more compartments.

13. A method of introducing oxygen into an aerobic organic material processor, comprising the steps of:
providing an aerobic organic material processor, comprising:
a tank having an outer wall, a top, a bottom, a first end, a second end, and a horizontal axis, the tank being divided into compartments distributed along the horizontal axis of the tank between the first end of the tank and the second end of the tank, each compartment being fluidly connected to the other compartments by a fluid passageway toward the bottom of the tank;
an organic material input positioned toward the first end of the tank;
an organic material output positioned toward the second end of the tank;
an agitator for agitating the organic material within the tank; and
providing fluid ports toward the top of the tank in communication with each of the compartments;
applying air under pressure to one of the fluid ports to cause air to migrate from compartment to compartment via the fluid passageway, thereby aerating organic material within the tank, with excess fluid being expelled through the other fluid ports.
